(19) United States
(12) Patent Application Publication
(10) Pub. No.: US 2024/0285832 A1
(43) Pub. Date: Aug. 29, 2024

(54) SCAFFOLD FOR TREATMENT OF TENDON OR LIGAMENT DISEASES BY INSERTING ATELOCOLLAGEN INTO POROUS POLYMER OF HYALURONIC ACID

(71) Applicants: UNIVERSITY-INDUSTRY COOPERATION GROUP OF KYUNG HEE UNIVERSITY, Yongin-si (KR); RESEARCH & BUSINESS FOUNDATION SUNGKYUNKWAN UNIVERSITY, Suwon-si (KR)

(72) Inventors: Sung Min RHEE, Seoul (KR); Jae Yun KIM, Suwon-si (KR); Pil Seon IM, Suwon-si (KR)

(73) Assignees: UNIVERSITY-INDUSTRY COOPERATION GROUP OF KYUNG HEE UNIVERSITY, Yongin-si (KR); RESEARCH & BUSINESS FOUNDATION SUNGKYUNKWAN UNIVERSITY, Suwon-si (KR)

(21) Appl. No.: 18/585,241

(22) Filed: Feb. 23, 2024

(30) Foreign Application Priority Data
Feb. 23, 2023 (KR) 10-2023-0024449

(51) Int. Cl. A61L 27/56; A61L 27/26; A61L 27/58

(52) U.S. Cl. CPC A61L 27/56; A61L 27/26; A61L 27/58; A61L 2430/10

(57) ABSTRACT

The present disclosure relates to a scaffold with atelocollagen inserted into a porous hyaluronic acid polymer for treatment and recovery of tendon or ligament diseases. The scaffold enhances the efficacy of hyaluronic acid and atelocollagen through slow and prolonged secretion at the suture site, thereby creating a synergistic effect. Additionally, the porous scaffold with high mechanical properties binds the suture site of a ruptured tendon and provides a microenvironment for the regeneration process of surrounding cells, thereby making it useful for the treatment and recovery of tendon or ligament diseases.

### CROSS-REFERENCE TO RELATED APPLICATION

A claim for priority under 35 U.S.C. § 119 is made to Korean Patent Application No. 10-2023-0024449 filed on Feb. 23, 2023 in the Korean Intellectual Property Office, the entire contents of which are hereby incorporated by reference.

### BACKGROUND

### 1. Technical Field

The present disclosure relates to a scaffold for the treatment of tendon or ligament diseases by inserting atelocollagen into a porous polymer of hyaluronic acid, and a method for manufacturing the same.

### 2. Description of Related Art

Regenerative medicine refers to the medical field that aims to replace or regenerate human cells, tissues, and organs to restore original functions thereof. Regenerative medicine related to musculoskeletal disorders is expected to increase from USD 4.57 billion in 2019 to USD 13.53 billion in 2024, growing at an annual average rate of 24.3%. In Korea, there is growing interest in regenerative medicine, such as the use of local injections to improve tendon healing.

Tendons are typically tough bands of fibrous connective tissue which connect muscles to bones. Tendon injuries generally lead to inflammation and degeneration or weakening of the tendon, ultimately causing tendon rupture. Tendon healing is a long and complex process that typically takes several months, and it may take about a year for the tissue to gradually transform from a fibrous form to a scar. Such scar tissue can lead to reduced elasticity and mobility of the tendon and an increased tendency for injury recurrence.

Hyaluronic acid (HA) preferably has a molecular weight of about 100,000 to 5,000,000, but can be used regardless of the molecular weight. Hyaluronic acid, a natural polymer, has anti-inflammatory properties and helps form the fibrocartilage layer, which is essential for tendon healing. Moreover, hyaluronic acid is biocompatible, facilitates cell movement during tissue formation, and is completely decomposed by enzymes in the body.

Collagen is hard protein that makes up bones, cartilage, teeth, tendons, the skin of animals, and the scales of fish, is also known as gelatin, and exists as a fibrous solid with a complex striated structure. Collagen is the most common structural protein in all mammals, occupying about 30% of total protein weight. It is known that there are about 20 types of collagen, with type 1 collagen making up the majority.

Atelocollagen is a type 1 collagen, supplementing the type 1 collagen that is a major component of tendons, thereby aiding in tendon healing, and reducing inflammation. Atelocollagen is preferably used with a molecular weight of about 1,000 to 20,000, but can be used regardless of the molecular weight. Atelocollagen is advantageous for cell adhesion during tissue formation, and maintains a physical skeleton during the manufacture of a scaffold.

However, the currently commercialized hyaluronic acid and/or atelocollagen injections are all in liquid type. However, in a case of being injected into the joint space expanded with physiological saline during suturing, the liquid type hyaluronic acid and/or atelocollagen do not remain only at the tendon suture site but flow down to the surrounding area.

Under the above background, the inventors of the present invention have created a porous scaffold with hyaluronic acid, and inserted atelocollagen into the porous scaffold and made the atelocollagen slowly secrete at the suture site over a long period, thereby enhancing the efficacy of hyaluronic acid and atelocollagen and confirming a synergistic effect. Additionally, the inventors of the present invention have bound the porous scaffold with high mechanical properties to the suture site of a ruptured tendon and provided a microenvironment for the regeneration process of surrounding cells, thereby enhancing the treatment and recovery effects for tendon or ligament diseases. Through the above, the present invention has been completed.

### SUMMARY

The present disclosure has been made to solve the above-mentioned problems occurring in the prior art, and in an aspect of the present disclosure, an object of the present disclosure is to provide a scaffold for treatment of tendon or ligament diseases, which includes: a porous hyaluronic acid cryogel; collagen immersed in pores of the porous hyaluronic acid cryogel; and a bioabsorbable copolymer fiber adhered on the first surface of the porous hyaluronic acid cryogel.

Another object of the present disclosure is to provide a method for manufacturing a scaffold for treatment of tendon or ligament diseases, which includes: operation i) inserting and cryogelating a solution containing hyaluronic acid and an initiator into a mold including bioabsorbable copolymer fibers to prepare a porous hyaluronic acid cryogel; and operation ii) immersing collagen into pores of the hyaluronic acid cryogel obtained in step i).

Advantages and features of the present disclosure and methods accomplishing the advantages and features will become apparent from the following detailed description of exemplary embodiments with reference to the accompanying drawings. However, the present disclosure is not limited to exemplary embodiment disclosed herein but will be implemented in various forms. The exemplary embodiments are provided so that the present disclosure is completely disclosed, and a person of ordinary skilled in the art can fully understand the scope of the present disclosure. Therefore, the present disclosure will be defined only by the scope of the appended claims.

Terms used in the specification are used to describe specific embodiments of the present disclosure and are not intended to limit the scope of the present disclosure. In the specification, the terms of a singular form may include plural forms unless otherwise specified. It should be also understood that the terms of ‘include’ or ‘have’ in the specification are used to mean that there is no intent to exclude existence or addition of other components besides components described in the specification. In the detailed description, the same reference numbers of the drawings refer to the same or equivalent parts of the present disclosure, and the term “and/or” is understood to include a combination of one or more of components described above. It will be understood that terms, such as “first” or “second” may be used in the specification to describe various components but are not restricted to the above terms. The terms may be used to discriminate one component from another component. Therefore, of course, the first component may be named as the second component within the scope of the present disclosure.

Unless otherwise defined, all terms (including technical and scientific terms) used herein have the same meaning as commonly understood by those skilled in the technical field to which the present disclosure pertains. It will be further understood that terms, such as those defined in commonly used dictionaries, should not be interpreted in an idealized or overly formal sense unless expressly so defined herein.

The present invention provides a scaffold for treatment of tendon or ligament diseases, which includes: a porous hyaluronic acid cryogel; collagen immersed in pores of the porous hyaluronic acid cryogel; and a bioabsorbable copolymer fiber adhered on the first surface of the porous hyaluronic acid cryogel.

The present invention provides a scaffold for the treatment of tendon or ligament diseases using a porous hyaluronic acid cryogel, namely, a hyaluronic acid-based porous cryogel.

The hyaluronic acid (HA) of the present invention is a polysaccharide with a molecular weight of 50,000 to 10,000,000 Da or more, which is a linear polymer comprising β-D-N-acetylglucosamine and β-D-glucuronic acid alternately combined. The hyaluronic acid is a basic substance of biological connective tissues, mainly distributed in the skin, synovial fluid of joints, vitreous body fluid of eyes, umbilical cord, and serum of mammals, cockscomb, etc., and also exists in the capsules of streptococcus and bacillus species. Natural hyaluronic acid illustrates polydispersity proportional to the molecular weight, but does not have interspecies specificity and does not have tissue or organ specificity. Accordingly, natural hyaluronic acid exhibits excellent biocompatibility when implanted or injected into the body, regardless of its origin.

The hyaluronic acid which is the natural polymer preferably has a molecular weight of about 100,000 to 5,000,000, but can be used regardless of the molecular weight. It is preferable that hyaluronic acid is included in a weight ratio of 70 to 95% in a mixed solution. If the content of hyaluronic acid is below 70%, the physical strength of the produced scaffold weakens, and if it exceeds 95%, it becomes unsuitable for handling when being mixed. The hyaluronic acid, as one of the components of cartilage, has biocompatibility, is advantageous for cell movement during tissue formation, and is completely decomposed by enzymes in the body.

In the field of tissue engineering, a scaffold is essential for providing a three-dimensional porous structure to support tissue formation. The scaffold provides at least one of the following: cell adhesion and movement; delivery and retention of cells and biochemical factors; diffusion of vital cell nutrients and expressed products; and specific mechanical and biological influences to modify the behavior of a cell stage. In the present invention, the scaffold can be described in combination with a support.

The cryogel in the present invention is a material with a highly variable porous structure and can take various forms. The cryogel can be produced using cryotropic gelation technology.

Cryotropic gelation, cryogelation, or cryostructuration in the present invention refers to a specific type of gel formation that occurs as a result of the freeze-thawing processing of a system potentially capable of gelation. An intrinsic feature of cryotropic gelation is the forced crystallization of a solvent like water. Cryotropic gelation is distinguished from cooling-induced gelation that occurs as the temperature decreases. A typical characteristic of cryotropic gelation is the ability to create an interconnected system of macropores. The size of the pores can be as large as several hundred microns. Cryogel often has a sponge-like form, which contrasts with the typical continuous monophase gel formed from the same precursor but generated above freezing temperatures.

The collagen in the present invention is a hard protein that makes up bones, cartilage, teeth, tendons, and skin of animals, and scales of fish, also known as gelatin, and exists as a fibrous solid. Under an electron microscope, collagen illustrates a complex striated structure. Collagen is the most frequent structural protein in all mammals, occupying about 30% of the total protein weight. It is known that there are about 20 types of collagen, with type 1 collagen making up the majority. Collagen has a structure that monomeric proteins of about 300 kda are cross-linked at specific sites through covalent bonds. Thus, collagen is insoluble and can form a characteristic fibrous form with high tensile strength. The constituent amino acids include glycine, proline, hydroxyproline, alanine, glutamic acid, etc., and among them, hydroxyproline, which is not found in other proteins, is high in content.

The collagen in the present invention has a structure in which three polypeptide strands are twisted together by hydrogen bonds. Collagen does not decompose in water, dilute acid, or dilute alkali, but when boiled, becomes gelatin in a single-strand structure and can dissolve. Collagen differs from gelatin in that collagen can achieve an appropriate viscosity without heating, so, can be easily produced through gelation. Furthermore, being a higher polymer than gelatin, collagen is closer to biological tissues, has greater bioactivity, and promotes healing when used on wounds.

The collagen in the present invention may be type 1 collagen.

The collagen in the present invention may be atelocollagen, bovine collagen or human collagen. It is preferable to use atelocollagen with a molecular weight of about 1,000 to 20,000, but atelocollagen can be used regardless of the molecular weight. Atelocollagen has the immunogenic telopeptide portion of collagen removed to eliminate factors that can cause an immune response in the body. It is advantageous for cell adhesion during tissue formation and can maintain a physical skeleton during scaffold production.

The copolymer in the present invention can be one or more selected from the group consisting of polylactide (PLA); poly(lactide-co-glycolide) (PLGA); polyanhydride; polyorthoester; poly(N-(2-hydroxypropyl) methacrylamide); poly(dl-lactide) (DLPLA); poly(l-lactide) (LPLA); poly(d-lactide) (DPLA); polyglycolide (PGA); poly(dioxanone) (PDO); poly(glycolide-co-trimethylene carbonate) (PGA-TMC); poly(l-lactide-co-glycolide) (PGA-LPLA); poly(dl-lactide-co-glycolide) (PGA-DLPLA); poly(1-lactide-co-dl-lactide) (LPLA-DLPLA); poly(glycolide-co-trimethylene carbonate-co-dioxanone) (PDO-PGA-TMC), poly(lactic acid-co-caprolactone) (PLACL), Poly-L-Lactic Acid (PLLA), and mixtures or copolymers thereof, but is not limited thereto.

The copolymer in the present invention may be poly(lactide-co-glycolide) (PLGA), but is not limited thereto. The copolymer is adhered only onto the first surface of the porous hyaluronic acid cryogel.

The porous hyaluronic acid cryogel in the present invention, due to low mechanical properties thereof, could be destroyed when regular sutures are used, so copolymer fibers are used, and in a specific embodiment of the present invention, a PLGA mesh is used.

In a specific embodiment of the present invention, when the scaffold was sutured without the PLGA mesh, it was observed that the scaffold crumbled during a scaffold insertion process, so it was difficult to stably suture the rabbit's deltoid. However, when the scaffold was sutured using the PLGA mesh, it was confirmed that the scaffold could be inserted stably without crumbling and then the deltoid could be sutured.

The tendon in the present invention refers to a fibrous tissue consisting of closely stacked parallel arrays of collagen fibers that connect muscles to bones. Healing of damaged tendons is a slow process and is often associated with scarring, which can result in tendons with defects that are normal or incapable of resuming original tendon functions.

The tendon damage in the present invention refers to the partial rupture, complete rupture, or degeneration of a tendon. Tendon damage is related with muscles, and can occur due to the transmission of strain to the tendon when there is a lack of flexibility in the muscles.

The tendon diseases may be chronic disorders or damages to tendons that gradually wear out due to overuse or aging, or tears, inflammations, or ruptures of tendons due to acute injuries, or separations of the tendon from the bone. Specifically, tendon diseases may be one or more selected from a group consisting of Achilles tendinitis, patellar tendinopathy, lateral epicondylitis, medial epicondylitis, plantar fasciitis, rotator cuff tendinopathy, tenosynovitis, tendinopathy, tendinitis, tenosynovitis, tendon injuries, and tendon avulsions, but are not limited thereto.

The ligament in the present invention refers to the connective tissue connecting bones to bones.

Ligament damages in the present invention refers to the partial rupture, complete rupture, or degeneration of the ligaments. Damage to a ligament can lead to abnormal movement axes of joints, and results in damage to internal joint structures, such as cartilage, and may potentially progress to arthritis.

The ligament diseases may be one or more selected from a group consisting of injuries to the cruciate ligaments, ankle ligaments, collateral ligaments, inflammations, ligament ruptures, and ligament sprains, but are not limited thereto.

The treatment in the present invention generally refers to achieving a desired pharmacological and/or physiological effects. Preferably, the effects mean effects in partially or completely treating tendon or ligament damage. That is, treatment may not only involve the improvement of symptoms or reduction of disease markers but also the cessation or resolution of symptoms that could be expected without any treatment. Beneficial or desired clinical outcomes can include alleviation of one or more symptoms, reduction in the severity of the disease, stabilized (i.e., not worsened) state of the disease, delay or resolution of disease progression, improvement or temporary relief of the disease state, and partial or total remission, but are not limited thereto.

The scaffold of the present invention can increase the gene expression of Col1 (Collagen 1) and Col3 (Collagen 3).

In a specific embodiment of the present invention, it was confirmed that the gene expression of Collagen 1 and Collagen 3 was highest in the group where the scaffold, injected with atelocollagen into the pores of the porous hyaluronic acid cryogel, was sutured over the rotator cuff suture site.

The scaffold can increase the stiffness of tendons or ligaments.

In a specific embodiment of the present invention, it was confirmed that the biomechanical results (stiffness) of the group with atelocollagen inserted into the porous hyaluronic acid polymer were superior. The stiffness of the scaffold in the present invention can be 30 to 60 N/mm, specifically 35 to 55 N/mm, and more specifically 42 N/mm, but is not limited thereto.

Additionally, the scaffold of the present invention can treat inflammation in tendons or ligaments and prevent re-rupture of tendons or ligaments.

The prevention in the present invention refers to suppressing the occurrence of a disease or a disorder in a subject who has not been diagnosed with diseases but is at risk of a tendon or ligament disease or disorder.

In addition, the present invention provides a method for method for manufacturing a scaffold for treatment of tendon or ligament diseases, comprising: operation i) inserting and cryogelating a solution containing hyaluronic acid and an initiator into a mold including bioabsorbable copolymer fibers to prepare a porous hyaluronic acid cryogel; and operation ii) immersing collagen into pores of the hyaluronic acid cryogel obtained in step i).

The hyaluronic acid, the cryogelation, the cryogel, the copolymer, the collagen, tendon, the tendon damage, the tendon disease, the ligament, the ligament damage, the ligament disease, the treatment, and the scaffold in the present invention are as described above.

The hyaluronic acid in the solution can be 0.1 to 3 weight %, specifically 0.5 to 2 weight %, and more specifically 1 weight %, but is not limited thereto. If the hyaluronic acid is less than 0.1 weight % in the solution, it can be easily decomposed since properties thereof may be significantly low, and if it exceeds 3 weight %, it may be difficult to form a porous scaffold.

The cryogelation in the present invention can be performed at −20 to −40° C. for 15 to 20 hours, specifically at −20 to −30° C. for 16 to 19 hours, and more specifically at −20° C. for 18 hours.

The porous hyaluronic acid cryogel in the present invention can be synthesized through cryogelation using hyaluronic acid methacrylate (HAMA) or thiolated hyaluronic acid as a precursor.

The copolymer in the present invention may be poly(lactide-co-glycolide) (PLGA), but is not limited thereto. The copolymer is adhered only onto the first surface of the porous hyaluronic acid cryogel.

In a specific embodiment of the present invention, the porous hyaluronic acid cryogel in the present invention, due to low mechanical properties thereof, could be destroyed when regular sutures are used, so a PLGA mesh is used.

The tendon diseases may be chronic disorders or damages to tendons that gradually wear out due to overuse or aging, or tears, inflammations, or ruptures of tendons due to acute injuries, or separations of the tendon from the bone. Specifically, tendon diseases may be one or more selected from a group consisting of Achilles tendinitis, patellar tendinopathy, lateral epicondylitis, medial epicondylitis, plantar fasciitis, rotator cuff tendinopathy, tenosynovitis, tendinopathy, tendinitis, tenosynovitis, tendon injuries, and tendon avulsions, but are not limited thereto.

The ligament diseases may be one or more selected from a group consisting of injuries to the cruciate ligaments, ankle ligaments, collateral ligaments, inflammations, ligament ruptures, and ligament sprains, but are not limited thereto.

### BRIEF DESCRIPTION OF THE DRAWINGS

FIG. 1 illustrates a process for preparing a porous hyaluronic acid cryogel.

FIG. 2 illustrates the characteristics of a porous scaffold with mechanical stability against compression.

FIG. 3 illustrates a process of preparing a porous hyaluronic acid cryogel with a PLGA mesh fixed on one side and the produced cryogel.

FIG. 4 illustrates a hyaluronic acid/atelocollagen scaffold produced by immersing atelocollagen into empty spaces of the porous hyaluronic acid cryogel.

FIG. 5 illustrates the stability of atelocollagen loaded in macropores of the cryogel.

FIG. 6 illustrates a hyaluronic acid/atelocollagen scaffold with atelocollagen immersed into the empty spaces (pores) of a large-area produced porous hyaluronic acid cryogel.

FIG. 7 illustrates the form of the porous hyaluronic acid scaffold with atelocollagen immersed therein.

FIG. 8 illustrates a rabbit model with rotator cuff tear, where the supraspinatus tendon is completely incised from a bone adhesion part and then sutured.

FIG. 9 illustrates the continuity and parallelism of collagen fibers confirmed through Masson's trichrome and Safranin 0 staining.

FIG. 10 illustrates the biomechanical results (stiffness) of experimental groups that atelocollagen was inserted into the porous hyaluronic acid polymer, wherein for Group 1, suture was performed after injection of 0.5 cc of physiological saline, for Group 2, only the porous hyaluronic acid cryogel was sutured over the rotator cuff suture site, for Group 3, suture was performed after injection of only atelocollagen, and for Group 4, the hyaluronic acid/atelocollagen scaffold, where atelocollagen was injected into the pores of the cryogel was sutured over the rotator cuff suture site.

FIG. 11 illustrates a comparison of results that scaffolds with and without PLGA mesh were sutured in a rabbit's deltoid muscle.

### DETAILED DESCRIPTION

Hereinafter, the present invention will be described in more detail through the following embodiments and experimental examples. However, the scope of the present invention is not limited to the following embodiments and experimental examples, and includes modifications of equivalent technical ideas.

### Embodiment 1. Manufacturing of Porous Support (Scaffold)

### 1-1. Preparation of Hyaluronic Acid and Atelocollagen

For hyaluronic acid, hyaluronic acid sodium salt from Sigma-Aldrich, which is a high molecular compound consisting of N-acetylglucosamine and glucuronic acid and is effective in cartilage formation, was used.

Atelocollagen is a collagenous component obtained by cutting off the telopeptides of both ends, which are antigenic substances causing hypersensitivity in type I collagen from pigs. For atelocollagen, Tendoregen from Ubiosis Co., Ltd., which is available domestically, was used.

### 1-2. Preparation of Porous Hyaluronic Acid Cryogel

The porous hyaluronic acid cryogel was synthesized through cryogelation using hyaluronic acid methacrylate (HAMA) as a precursor. That is, for 18 hours at −20° C., by using 0.4 wt % ammonium persulfate (APS) and 0.1 wt % N,N,N′,N′-tetramethylethylenediamine (TEMED) as initiators, cryogelation of hyaluronic acid methacrylate was performed through cryo-polymerization by free radical polymerization at a temperature below the freezing point of water. The cryogel could recover a shape thereof after a removal of compressive force, thereby indicating mechanical stability against compression. However, the cryogel is not suitable for suturing.

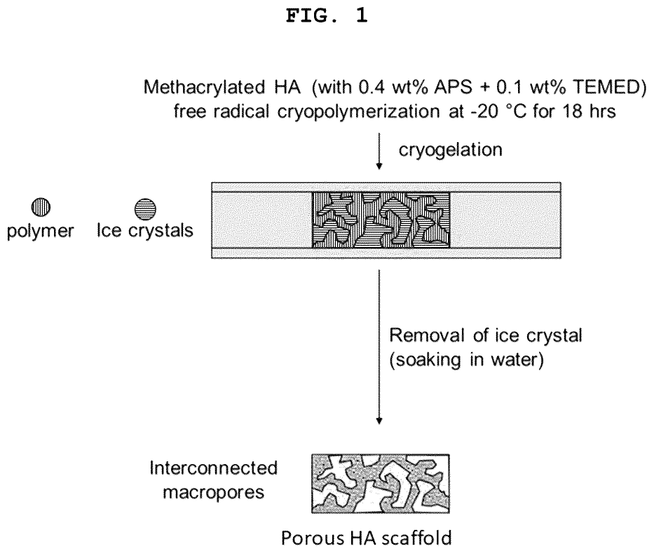

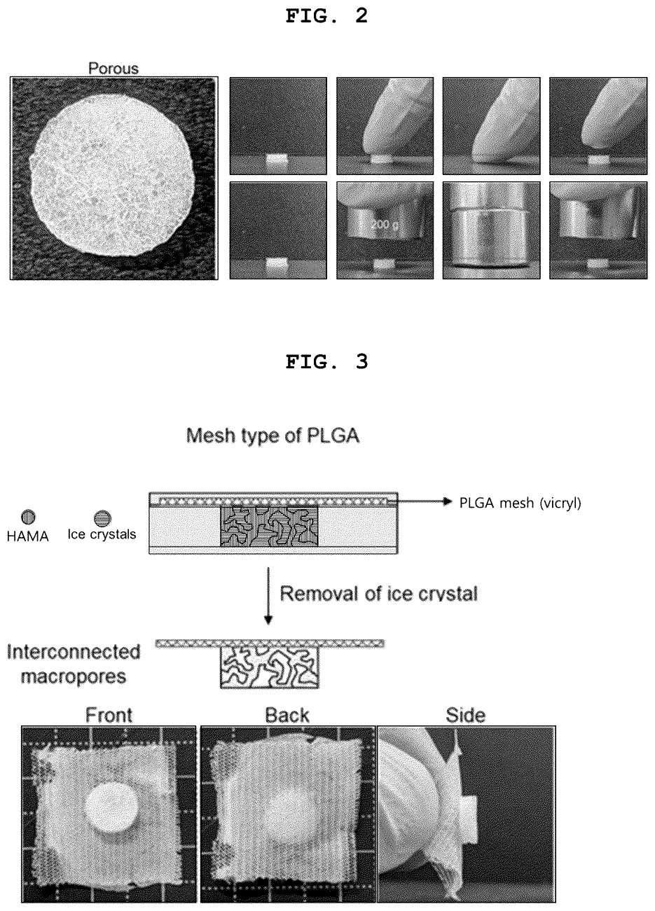

Therefore, ice crystals formed during the cryo-polymerization process were removed to create a macroporous structure (FIG. 1). The macroporous structure, which is an interconnected structure in which pores are all connected, has elasticity and resilience, thus making it suitable for carrying and releasing highly viscous atelocollagen (FIG. 2).

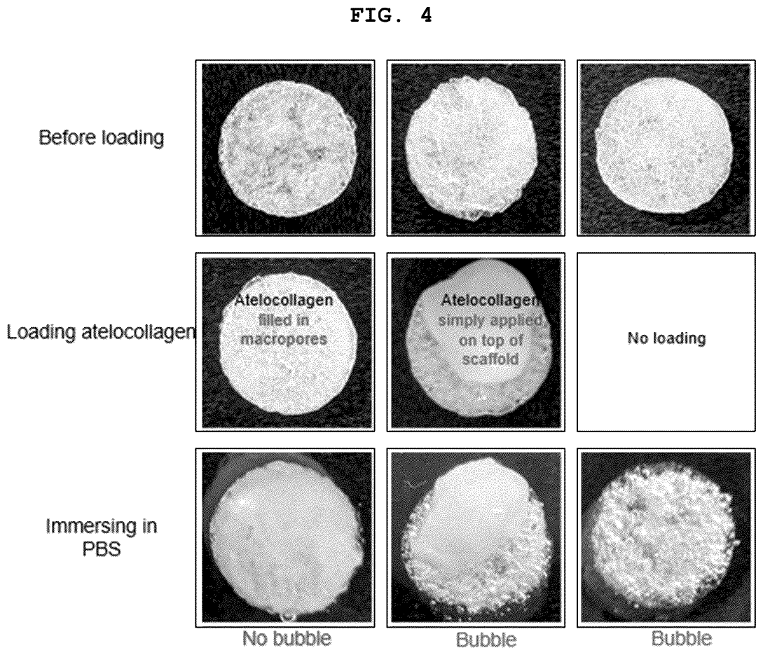

However, if only the porous hyaluronic acid cryogel is used, the scaffold may easily break during suturing. To solve such a problem, a porous hyaluronic acid cryogel with a PLGA mesh fixed on one side was prepared (FIG. 3). That is, a solution containing 1 wt % methacrylated hyaluronic acid and initiator was cryogelated, and PLGA mesh was adhered onto one side to prepare a hyaluronic acid-based porous cryogel.

Atelocollagen Immersion into Porous Cryogel

Atelocollagen was immersed into empty spaces (pores) of the porous hyaluronic acid cryogel to prepare a hyaluronic acid/atelocollagen scaffold, not just a simple mixture of atelocollagen and hyaluronic acid. The absence of bubbles indicates that the macropores are well filled with atelocollagen, and bubbles represent the empty holes in the scaffold (FIG. 4).

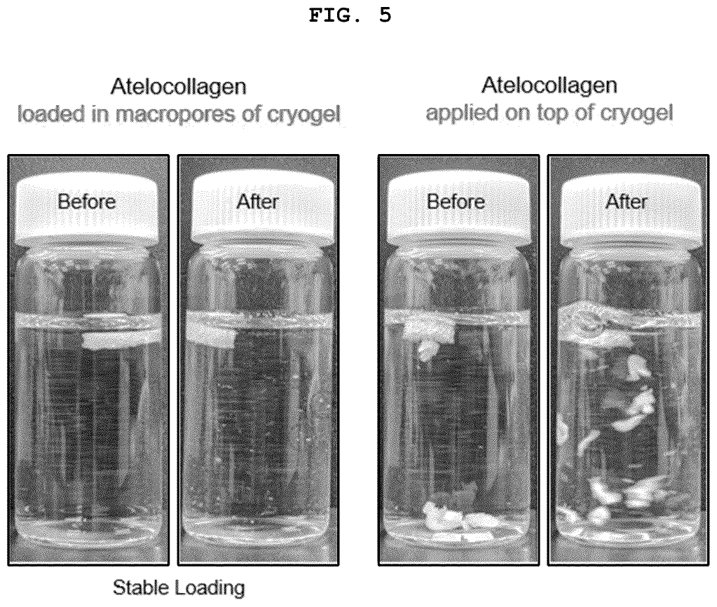

When the hyaluronic acid cryogel/atelocollagen is vigorously shaken in distilled water and ultrasonically treated for 10 seconds, the stability of atelocollagen loaded into the macropores of the cryogel can be observed (FIG. 5). It can be confirmed that since atelocollagen is stably loaded inside the pores of the porous hyaluronic acid scaffold, the atelocollagen does not fall off or are not released out even when shaken. In contrast, if atelocollagen is simply placed on the surface of the scaffold or pressed onto the scaffold, atelocollagen is not properly loaded and falls off.

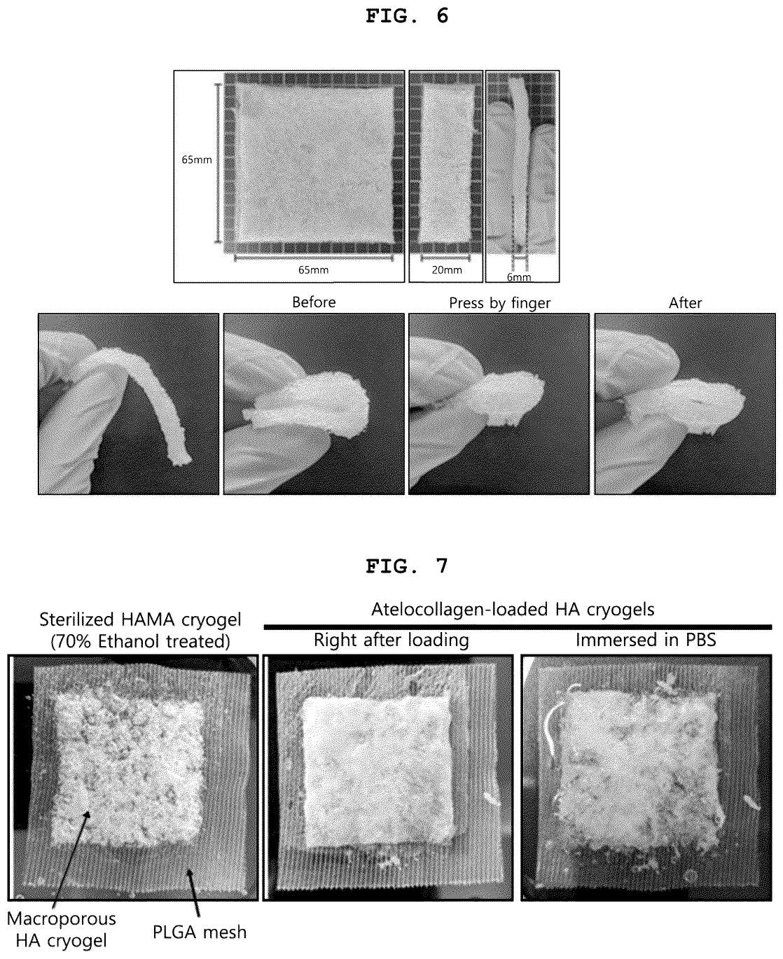

The atelocollagen can also be prepared in large areas, specifically prepared in 65 mm×65 mm×6 mm (FIG. 6).

The atelocollagen-immersed hyaluronic acid cryogel produced according to Embodiments 1-1 to 1-3 is as illustrated in FIG. 7, and FIG. 7 illustrates sterilized hyaluronic acid cryogel treated with 70% of ethanol right after loading, and sterilized hyaluronic acid cryogel immersed in PBS.

### Embodiment 2. Statistical Basis by Calculation of the Number of Animals

Experiments were conducted in four groups. Each group was extracted at 12 weeks after suturing, and histological and biomechanical tests were conducted. The number of experimental animals illustrating a significant difference in maximum burst strength was determined through a power analysis with an average maximum burst strength of 90N, a standard deviation of 40N, an alpha error of 0.05, a beta error of 0.2, and a dropout rate of 25%. In the experiment, 8 animals per group, totaling 32 animals, were used.

### Embodiment 3. Animal Experiment

The experimental rabbits were anesthetized with 5 mg/kg IM Alfaxan and 5-10 mg/kg IM xylazine. After shaving both shoulder joints, antibiotics were administered intramuscularly, and the animals were placed in the prone position on the operating table. The skin was disinfected, and only the surgical area was exposed using sterilized drapes. A 3 cm lateral incision was made on the outside of the shoulder joint. The supraspinatus tendon was exposed, and then, an adhesion site on the greater tubercle of the humerus was identified.

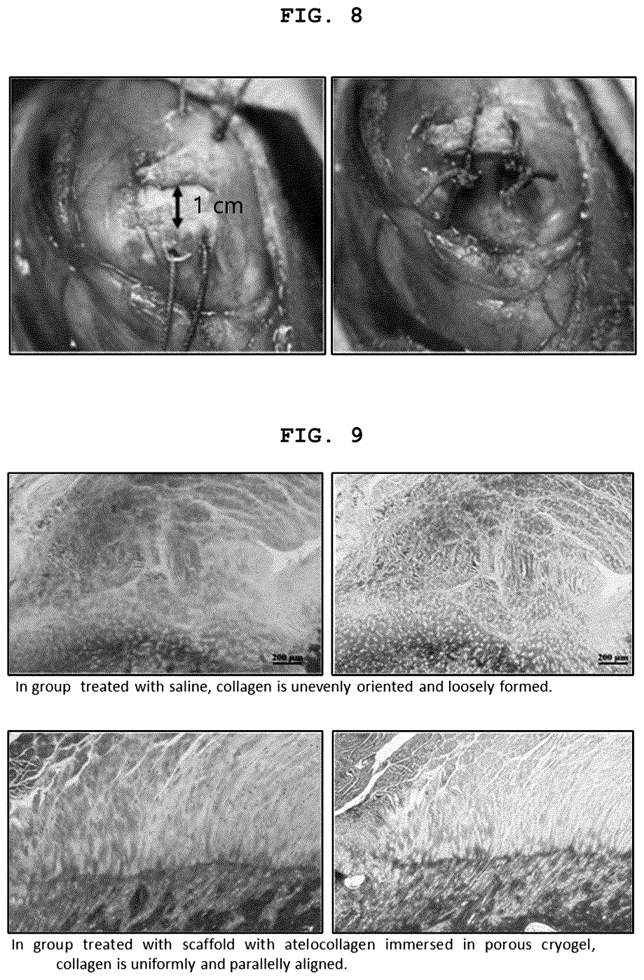

Both supraspinatus tendons were sharply separated from the greater tubercle using a scalpel, and completely incised so that no tendon or fibrocartilage tissue remained on the greater tubercle. To create a chronic rotator cuff tear model, suturing was performed after six weeks from the creation of the tear model. Bone tunneling suturing technique was used at the greater tubercle site to form a bone tunnel, and the supraspinatus tendon suturing was performed by passing two strands of suture (FIG. 8).

During the suturing, the corresponding substances for the experiment were injected. Afterward, the skin was sutured, antibiotics were administered once postoperatively, and the rabbits were allowed to move freely. Analgesics were subcutaneously injected for two days post-surgery for pain control. For three days from the surgery, Ketoprofen, an NSAID, was administered at 1-3 mg/kg IM twice daily, and if necessary, additional injections of one to two doses were performed to minimize pain. A neck collar was applied to prevent the animals from biting or licking the surgical site, and then, the animals were otherwise allowed to move freely and consume water and food.

In the present invention, through animal experiments, the effectiveness of healing rotator cuff by a scaffold in which atelocollagen was injected into the pores of the porous hyaluronic acid cryogel was analyzed. In the chronic rotator cuff tear model after six weeks from the tear, for Group 1, suture was performed after injection of 0.5 cc of physiological saline, for Group 2, only the porous hyaluronic acid cryogel was sutured over the rotator cuff suture site, for Group 3, suture was performed after injection of only atelocollagen, and for Group 4, the scaffold, where atelocollagen was injected into the pores of the cryogel was sutured over the rotator cuff suture site.

### Embodiment 4. Post-Surgery Evaluation

Twelve weeks post-suturing, all groups were anesthetized with 5 mg/kg IM Alfaxan and 5 to 10 mg/kg IM xylazine in the same way, and then, were euthanized through intravenous administration of 2 mmol/kg potassium chloride.

The surgical site was dissected to expose both supraspinatus tendons. The supraspinatus muscle of both sides, including 5 cm distal of the humerus, was extracted. A portion of the right tendon tissue was collected, and then, for gene expression analysis, placed in a cryotube and immediately frozen in liquid nitrogen.

The remaining right supraspinatus muscle was transversely cut 1 cm proximal from the bone-tendon junction, fixed in 10% buffered formalin (pH 7.4), and embedded in paraffin for histological examination. The left supraspinatus muscle underwent a biomechanical test. A transverse incision was made 5 cm proximal from the muscle-tendon junction, and then, was made 5 cm distal of the humerus.

For the biomechanical tensile strength measurement, a universal testing machine (Shimadzu, AGS-X) was used. The muscle and tendon were securely fixed to an upper jig, and the humerus was firmly fixed to a lower holding device to prevent slippage of the tissue during tension measurement.

### Experimental Example 1. Measurement of Gene Expression in Muscle and Tendon

Upon extraction at 12 weeks post-suturing in all groups, a portion of the tendon was collected from the right supraspinatus muscle to confirm the gene expression of Col1 and Col3, which are expressed in collagen I and III, respectively, Sderaxis expressed during tendon formation, Sox9 expressed during cartilage formation, Aggrecan, and BMP-2 expressed during bone formation.

RNA was extracted from the tissue using a RNeasy Mini Kit, followed by synthesis and amplification of cDNA. Finally, real-time polymerase chain reaction (real-time PCR) was performed using qRT-PCR: Power SYBR® Green PCR Master Mix to confirm the expression of each RNA.

As a result, it was confirmed that the gene expression of Collagen 1 and Collagen 3 was highest in Group 4, where the scaffold with atelocollagen injected into the pores of the porous hyaluronic acid cryogel was sutured above the rotator cuff suturing site (Table 1).

TABLE 1

| | Group 1 | Group 2 | Group 3 | Group 4 | P value |
|---|---|---|---|---|---|
| Collagen1 | 1.1 ± 0.6 | 1.0 ± 0.3 | 1.2 ± 0.7 | 1.3 ± 0.9 | 0.947 |
| Collagen3 | 1.3 ± 0.9 | 1.2 ± 0.8 | 1.3 ± 1.0 | 1.3 ± 0.9 | 0.971 |
| SOX | 1.1 ± 0.4 | 1.0 ± 0.3 | 1.2 ± 0.6 | 1.1 ± 0.5 | 0.750 |
| SCX | 1.1 ± 0.5 | 1.0 ± 0.4 | 1.1 ± 0.4 | 1.1 ± 0.4 | 0.976 |

It suggests that the partial suturing using the hyaluronic acid/atelocollagen scaffold according to the present invention, for rotator cuff tears, is very effective.

### Experimental Example 2. Histological Analysis

At 12 weeks post-suturing in all groups, the right supraspinatus muscle were extracted for histological analysis of the bone-tendon junction. Hematoxylin and Eosin (H&E) staining, Masson's trichrome staining, and Safranin 0 staining were performed.

The collagen arrangement at the bone-tendon junction was observed under a microscope (Olympus BX51, Olympus, Tokyo, Japan) at 100× magnification. Images were captured using the AxioVision v. 4.4 (Carl Zeiss) program, and then, the vascularity, cellularity, collagen fiber continuity, and the proportion of fibers parallel to the tendon-to-bone interface were measured.

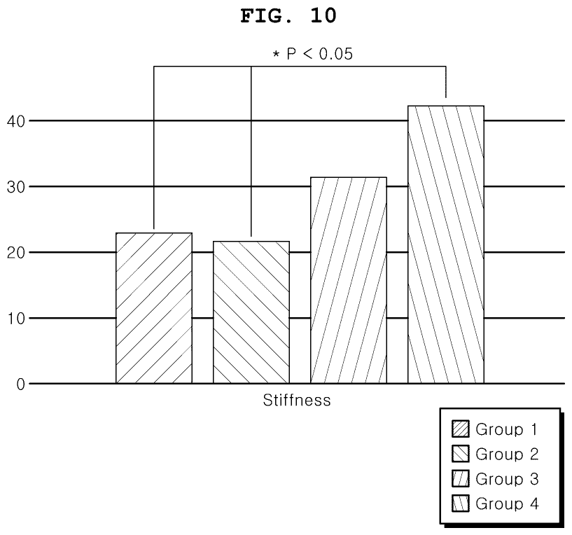

Collagen fibers were stained blue in Masson's trichrome staining to identify continuity and parallelism. Additionally, the formation of fibrocartilage was identified through Safranin 0 staining (FIG. 9).

### Experimental Example 3. Biomechanical Tensile Strength Measurement

A universal testing machine (Shimadzu, AGS-X) was used to measure the biomechanical tensile strength. The muscle and tendon were firmly fixed to an upper jig, and the humerus was firmly fixed to a lower holding device to prevent tissue slippage during tension.

The tendon was stretched according to an original anatomical direction thereof. Preconditioning was performed at a loading rate of 15 N/s from an initial 5N to a maximum of 50N, and then, measurement up to the point where the tensile strength dropped by 40% at a preload of 5N and a speed of 1 mm/s was performed.

Data on the maximum load and strength at rupture were automatically saved through a data acquisition system connected to the computer.

The result variables are as follows: i) the rupture site and mode (suture pullout or tendon mid-substance tear) as determined by a tensile test; ii) the maximal load at failure during tear; and iii) the stiffness of the tendon.

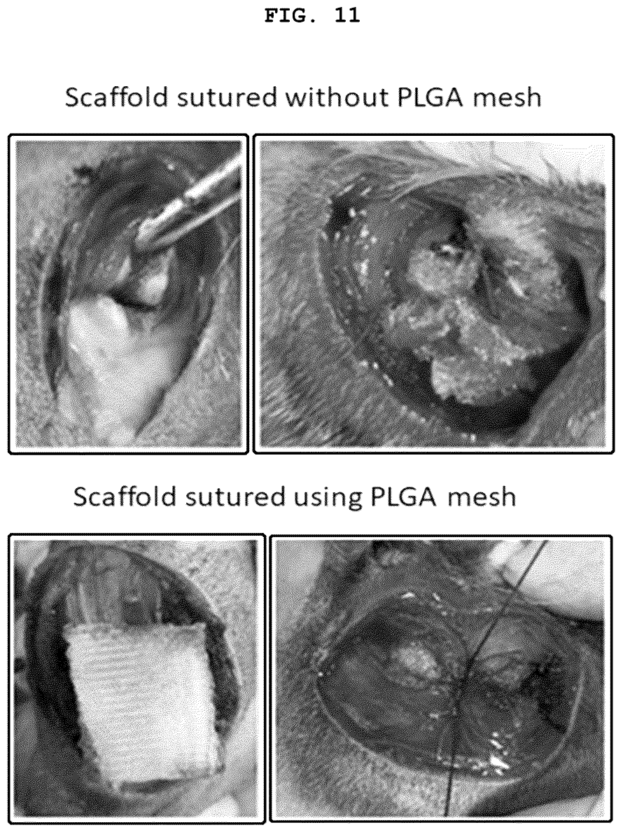

As a result, it was confirmed that the biomechanical results (stiffness) of the group 4 with atelocollagen inserted into the porous hyaluronic acid polymer were superior. (Table 2 and FIG. 10).

TABLE 2

| | Group 1 | Group 2 | Group 3 | Group 4 | P |
|---|---|---|---|---|---|
| | (n = 6) | (n = 6) | (n = 7) | (n = 8) | value |
| Load to | 138.6 ± 38.7 | 164.5 ± 49.5 | 158.4 ± 76.1 | 191.3 ± 101.4 | 0.762 |
| failure | | | | | |
| (N) | | | | | |
| Load to | 28.3 ± 7.7 | 36.6 ± 13.2 | 37.8 ± 18.7 | 46.6 ± 26.3 | 0.538 |
| failure | | | | | |
| (N/kg) | | | | | |
| Stiffness | 22.8 ± 6.9 | 21.5 ± 3.9 | 31.2 ± 10.8 | 42.2 ± 7.5 | 0.005 |
| (N/mm) | | | | | |

### Experimental Example 4. Statistics and Analysis

For variables with continuous values, the Kruskal-Wallis test and post-hoc Mann-Whitney test were used to analyze differences in gene expression, histological examination, and biomechanical maximum rupture load and stiffness among the groups. The rupture site and mode with non-continuous variables were compared among the groups using Fisher's exact test. All statistical analyses were performed using the SPSS program (version 15.0, SPSS Inc., Chicago, IL), and a P-value of less than 0.05 was considered statistically significant.

The above description is only exemplary, and it will be understood by those skilled in the art that the disclosure may be embodied in other concrete forms without changing the technological scope and essential features. Therefore, the above-described embodiments should be considered only as examples in all aspects and not for purposes of limitation.

### Advantageous Effects

The present invention provides a scaffold with atelocollagen inserted into a porous hyaluronic acid polymer for treatment and recovery of tendon or ligament diseases. The scaffold of the present invention enhances the efficacy of hyaluronic acid and atelocollagen through slow and prolonged secretion at the suture site, thereby creating a synergistic effect. Additionally, the porous scaffold with high mechanical properties binds the suture site of a ruptured tendon and provides a microenvironment for the regeneration process of surrounding cells, thereby making it useful for the treatment and recovery of tendon or ligament diseases.

1. A scaffold for treatment of tendon or ligament diseases, comprising:
a porous hyaluronic acid cryogel;
collagen immersed in pores of the porous hyaluronic acid cryogel; and
a bioabsorbable copolymer fiber adhered on the first surface of the porous hyaluronic acid cryogel.

2. The scaffold according to claim 1, wherein the collagen is type 1 collagen.

3. The scaffold according to claim 1, wherein the collagen is atelocollagen, bovine collagen or human collagen.

4. The scaffold according to claim 1, wherein the copolymer is one or more selected from the group consisting of polylactide (PLA); poly(lactide-co-glycolide) (PLGA); polyanhydride; polyorthoester; poly(N-(2-hydroxypropyl) methacrylamide); poly(dl-lactide) (DLPLA); poly(l-lactide) (LPLA); poly(d-lactide) (DPLA); polyglycolide (PGA); poly(dioxanone) (PDO); poly(glycolide-co-trimethylene carbonate) (PGA-TMC); poly(l-lactide-co-glycolide) (PGA-LPLA); poly(dl-lactide-co-glycolide) (PGA-DLPLA); poly(l-lactide-co-dl-lactide) (LPLA-DLPLA); poly(glycolide-co-trimethylene carbonate-co-dioxanone) (PDO-PGA-TMC), poly(lactic acid-co-caprolactone) (PLACL), Poly-L-Lactic Acid (PLLA), and mixtures or copolymers thereof.

5. The scaffold according to claim 1, wherein the copolymer is poly(lactide-co-glycolide) (PLGA).

6. The scaffold according to claim 1, wherein tendon diseases are one or more selected from a group consisting of Achilles tendinitis, patellar tendinopathy, lateral epicondylitis, medial epicondylitis, plantar fasciitis, rotator cuff tendinopathy, tenosynovitis, tendinopathy, tendinitis, tenosynovitis, tendon injuries, and tendon avulsions, and
wherein ligament diseases are one or more selected from a group consisting of injuries to the cruciate ligaments, ankle ligaments, collateral ligaments, inflammations, ligament ruptures, and ligament sprains.

7. The scaffold according to claim 1, wherein the scaffold obtains reinforcement of tendons or ligaments.

8. The scaffold according to claim 1, wherein the scaffold treats inflammation in tendons or ligaments.

9. The scaffold according to claim 1, wherein the scaffold prevents re-rupture of tendons or ligaments.

10. A method for manufacturing a scaffold for treatment of tendon or ligament diseases comprising:
operation i) inserting and cryogelating a solution containing hyaluronic acid and an initiator into a mold including bioabsorbable copolymer fibers to prepare a porous hyaluronic acid cryogel; and
operation ii) immersing collagen into pores of the hyaluronic acid cryogel obtained in step i).

11. The method according to claim 10, wherein the hyaluronic acid in the solution is 0.1 to 3 weight %.

12. The method according to claim 10, wherein the porous hyaluronic acid cryogel uses hyaluronic acid methacrylate (HAMA) or thiolated hyaluronic acid as a precursor.

13. The method according to claim 10, wherein the copolymer is one or more selected from the group consisting of polylactide (PLA); poly(lactide-co-glycolide) (PLGA); polyanhydride; polyorthoester; poly(N-(2-hydroxypropyl) methacrylamide); poly(dl-lactide) (DLPLA); poly(l-lactide) (LPLA); poly(d-lactide) (DPLA); polyglycolide (PGA); poly(dioxanone) (PDO); poly(glycolide-co-trimethylene carbonate) (PGA-TMC); poly(l-lactide-co-glycolide) (PGA-LPLA); poly(dl-lactide-co-glycolide) (PGA-DLPLA); poly(l-lactide-co-dl-lactide) (LPLA-DLPLA); poly(glycolide-co-trimethylene carbonate-co-dioxanone) (PDO-PGA-TMC), poly(lactic acid-co-caprolactone) (PLACL), Poly-L-Lactic Acid (PLLA), and mixtures or copolymers thereof.

14. The method according to claim 10, wherein the copolymer is poly(lactide-co-glycolide) (PLGA).

15. The method according to claim 10, wherein the collagen is type 1 collagen.

16. The method according to claim 10, wherein the collagen is atelocollagen, bovine collagen or human collagen.

17. The method according to claim 10, wherein the cryogelation is performed at −20 to −40° C. for 15 to 20 hours.

18. The method according to claim 10, wherein the copolymer fiber is adhered only on a first surface of the porous hyaluronic acid cryogel.

19. The method according to claim 10, wherein tendon diseases are one or more selected from a group consisting of Achilles tendinitis, patellar tendinopathy, lateral epicondylitis, medial epicondylitis, plantar fasciitis, rotator cuff tendinopathy, tenosynovitis, tendinopathy, tendinitis, tenosynovitis, tendon injuries, and tendon avulsions, and
wherein ligament diseases are one or more selected from a group consisting of injuries to the cruciate ligaments, ankle ligaments, collateral ligaments, inflammations, ligament ruptures, and ligament sprains.
